(19) United States
(12) Patent Application Publication
(10) Pub. No.: US 2008/0038381 A1
(43) Pub. Date: Feb. 14, 2008

(54) APPLICATION OF GREEN TEA EXTRACT AND ITS MAJOR COMPONENTS IN KELOID SCAR THERAPY

(75) Inventors: Anh D. Le, Los Angeles, CA (US); Qunzhou Zhang, South Pasadena, CA (US)

Correspondence Address: HOGAN & HARTSON L.L.P., 1999 AVENUE OF THE STARS, SUITE 1400, LOS ANGELES, CA, 90067, US

(73) Assignee: UNIVERSITY OF SOUTHERN CALIFORNIA, Los Angeles, CA (US)

(21) Appl. No.: 11/767,328

(22) Filed: Jun. 22, 2007

Related U.S. Application Data
(60) Provisional application No. 60/816,077, filed on Jun. 22, 2006.

(51) Int. Cl. A61K 36/82; A61K 31/352; A61P 17/02

(52) U.S. Cl. 424/729; 514/456

(57) ABSTRACT

A composition comprising a green tea extract (GTE) or its major component, (−)-epigallocatechin-3-gallate (EGCG). Also disclosed is a method of using the composition for keloid scar therapy.

### CROSS-REFERENCE TO RELATED APPLICATIONS

This application claims an invention which was disclosed in Provisional Application No. 60/816,077, filed Jun. 22, 2006. The benefit under 35 USC §119(e) of the United States provisional application is hereby claimed. The above priority application is hereby incorporated herein by reference.

### STATEMENT REGARDING FEDERALLY SPONSORED RESEARCH AND DEVELOPMENT

The present invention is made, at least in part, with the support of NIAMS/NIH grant AR47359 and P20 R011145. The government has certain rights in the invention.

### FIELD OF THE INVENTION

The invention pertains to the field of keloid scar therapy. More particularly, the invention pertains to a therapeutic composition comprising a green tea extract or a major component thereof. The invention also pertains to method of using the composition for keloid therapy.

### BACKGROUND OF THE INVENTION

Keloids are fibrous overgrowth induced by cutaneous injury (Tuan et al, 1998; Niessen et al., 1999). Clinically, keloids behave like benign dermal fibro-proliferative tumors as they continue to grow and extend beyond the confines of the original wound margins, without evidence of spontaneous regression as observed in hypertrophic scars (Rockwell et al., 1989; Ehrlich et al., 1994). Histologically, keloids and hypertrophic scars differ from normal skin and normal scars by their rich vasculature (Lee et al., 2004), high density of mesenchymal cells, a thickened epidermal cell layer, increased infiltration of inflammatory cells, including lymphocytes, mast cells and macrophages (Amadeu et al., 2003), and abundant deposition of extracellular matrix (ECM) (Rockwell et al., 1989; Niessen et al., 1999).

The underlying mechanism of keloid formation is still poorly understood, although abnormality in collagen synthesis leading to an imbalance in ECM metabolism has been recognized as an essential factor in the pathogenesis of keloid, as well as in several other fibrotic diseases (Niessen et al., 1999; Myllyharju et al., 2001).

Although clinically benign, the raised appearance of keloid scars are cosmetically undesirable. Unfortunately, surgical removal of keloid scars may stimulate further growth of the scars, hence, most people are told that they are untreatable.

Therefore, there still exists a need for a method to treat keloid scars.

### SUMMARY OF THE INVENTION

In view of the above, it is one object of the present invention to provide a method for treating keloid scars. In accordance with the objective of the present invention, there is provided a pharmaceutical composition capable of treating keloid scars and a method for treating keloid scars using the pharmaceutical composition.

In one aspect, the pharmaceutical composition of the present invention comprises a green tea extract or a major component thereof.

In another aspect, the present invention also provides a method for preventing keloid scars in a patient, comprising applying a pharmaceutical composition containing green tea extract or epigallocatechin-3-gallate to a fresh skin wound.

In yet another aspect, the present invention also provides a method for treating keloid scars, comprising surgically removing an existing keloid scar and then applying a pharmaceutical composition according to the present invention to the scared area.

Other aspects and advantages of the invention will be apparent from the following description and the appended claims.

### BRIEF DESCRIPTION OF THE DRAWINGS

FIG. 1 HMC-1 cells stimulate type I collagen expression in fibroblasts derived from keloids (KFs) and their corresponding peripheral normal skins (NSK). (a) KFs or NSK (1×10⁵/well) were directly co-cultured with increasing numbers of HMC-1 cells in a 6-well plate under normal culturing condition for 24 h. Equal amount of cell lysates (100 μg) was subjected to Western blot analysis with an anti-type I collagen antibody as described in Materials and Methods. (b) Densitometric analysis of results from A. (c) Dual-color immunofluorescent staining of type I collagen (Green) in keloid fibroblasts and c-kit on mast cells (Red). Co-cultured cells were fixed in cold methanol:acetone (1:1) and stained with mouse monoclonal anti-type I collagen antibody and rabbit polyclonal anti-c-kit antibody followed by incubation with Alexa Fluor®568 conjugated goat anti-rabbit IgG and Alexa Fluor®488 conjugated goat anti-mouse IgG. Representative results from three independent experiments are shown.

FIG. 2 Co-culture with HMC-1 cells activates multiple signaling pathways in keloid fibroblasts. Keloid fibroblasts were directly co-cultured with the same number of HMC-1 cells for different time intervals, and equal amount of cell lysates were subjected to Western blot analysis with various antibodies as described in Materials and Methods. Representative results from three independent experiments are shown.

FIG. 3 Involvement of PI-3k/Akt/mTOR and p38 MAPK pathways in mast cell stimulated type I collagen production in keloid fibroblasts. (a, c & e) Keloid fibroblasts (KFs) were pretreated with different concentrations of various inhibitors followed by direct co-culture with the same number of HMC-1 cells under normal culturing condition for 24 h. Equal amount of cell lysates (100 μg) was subjected to Western blot analysis with an anti-type I collagen antibody as described in Materials and Methods. (b, d & f) Densitometric analysis of results from a, c & e, respectively. The results represent three independent experiments and expressed as the mean ±SD.

FIG. 4 GTE and EGCG inhibit mast cell-stimulated type I collagen production in keloid fibroblasts. (a) Keloid fibroblasts (KFs) were pretreated with different concentrations of GTE or EGCG for 1 h followed by direct co-culture with the same number of HMC-1 cells under normal culturing condition for 24 h. Equal amount of cell lysates (100 μg) was subjected to Western blot analysis with an anti-type I collagen antibody as described in Materials and Methods. (b) Denssitometric analysis of results from a. (c) KFs were cultured in the presence of different concentrations of GTE or EGCG for 24 h, and type I collagen levels were determined by Western blot. (d) Denssitometric analysis of results from c. (e) Dual-color immunofluorescent staining of type I collagen (Green) in keloid fibroblasts and c-kit on mast cells (Red). Keloid fibroblasts (KFs) were pretreated with 80 μg/ml GTE for 1 h followed by co-cultured with HMC-1 cells (1:1) for 16 h. Then the co-cultured cells were fixed in cold methanol:acetone (1:1) and stained with mouse monoclonal anti-type I collagen antibody and rabbit polyclonal anti-c-kit antibody followed by incubation with Alexa Fluor®568 conjugated goat anti-rabbit IgG and Alexa Fluor®488 conjugated goat anti-mouse IgG. (f) RT-PCR analyses of pro-□1 (I) and pro-□2 (I) gene mRNA levels in the co-cultured keloid fibroblasts and HMC-1 cells after treatment with GTE, EGCG or various specific kinase inhibitors for 24 h. (g) KFs or HMC-1 ells were treated with various concentrations of GTE or EGCG for 24 h under normal culturing conditions and cell viability was assayed using MTT method. The percentage of viable cells represented the mean ±SD from three replicate experiments. The results represent three independent experiments and expressed as the mean ±SD.

FIG. 5 Effects of GTE and EGCG on mast cell-stimulated activation of ERK1/2 and Akt, phosphorylation of p70S6K and 4E-BP-1 in keloid fibroblasts. Keloid fibroblasts (KFs) were pretreated with different concentrations of GTE or EGCG, or various kinase inhibitors for 1 h, followed by direct co-culture with the same number of HMC-1 cells under normal culturing condition for 1 h. Equal amount of cell lysates was subjected to Western blot analysis with an antibodies against ERK1/2 (a), p38 MAPK (b), Akt, p70S6K and 4E-BP1 (c) as described in Materials and Methods. Data presented are representative of results from 3 independent experiments.

### DETAILED DESCRIPTION

Keloids is a chronic fibroproliferative disease. It characterized by excessive collagen deposition, and is prone to recurrence (human type I collagen comprises of two α1 (I) chains and one α2 (I) chain, which are derived from pro-COL1A1 and pro-COL1A2 genes, respectively (Raghow, 1994; Ghosh, 2002). It is the major component of ECM in skin, bone and ligaments). To date, the molecular mechanisms underlying the excessive collagen deposition remain largely unknown. Meanwhile, even though a number of treatment modalities have been employed to overcome keloid or to relieve its symptoms, there is no one modality that is always successful.

The inventors of the present invention have discovered that mast cell (MCs) co-cultures with HMC-1 cells substantially stimulates type I collagen synthesis in keloid fibroblasts.

It was also discovered that mast cell co-culture with HMC-1 cells lead to the activation of ERK1/2, PI-3K/Akt, and p38 MAPK signaling pathways. Interestingly, blockade of PI-3K/Akt, mTOR and p38 MAPK pathways by pretreatment with specific inhibitors significantly attenuates HMC-1 cell-stimulation for type I collagen synthesis in keloid fibroblasts.

Mast cells (MCs) are tissue dwelling cells containing prominent cytoplasmic granules. MC hyperplasia and activation have been implicated in the pathogenesis of several chronic inflammatory diseases such as autoimmune diseases, aberrant wound healing, idiopathic lung fibrosis, scleroderma, liver fibrosis, inflammatory bowel diseases such as Crohn's disease, and rheumatoid arthritis (Farrel et al., 1995; Cairns et al., 1997; Noli et al., 2001; Benoist et al., 2002; Puxeddu et al., 2003; Wooley, 2003; Nigrovic et al., 2005). Several studies have implicated a functional link between mast cells and abnormal skin wound healing (Choi et al., 1987), during which mast cells undergo significant qualitative and quantitative changes, leading to prolonged inflammation and altered proliferation dynamics (Artuc et al., 1999; Huttunen et al., 2000). Such changes in mast cells have been observed in keloids and hypertrophic scars, indicating an important role of mast cells and their mediators in the pathogenesis of keloid and hypertrophic scarring (Kischer et al., 1978; Craig et al., 1986; Smith et al., 1987; Lee et al., 1995; Zhang et al., 2006). However, the specific signaling pathways that are involved in mast cell-stimulated collagen synthesis remain largely unknown.

The inventors of the present invention have unexpectedly discovered that both green tea extract (GTE) and its major component (−)-epigallocatechin-3-gallate (EGCG) significantly suppresses mast cell-stimulated type I collagen expression in keloid fibroblasts. The results also indicate that the inhibitory effects of GTE and EGCG on type I collagen expression in keloid fibroblasts appear to be mediated via the phosphatidylinositol-3-kinase (PI-3K)/Akt/mTOR (mammalian target of rapamycin) signaling pathways.

These unique findings provide further understanding of the molecular mechanisms leading to the excessive collagen deposition in keloids and the anti-fibrogenic mechanisms of GTE and EGCG, and help to delineate further targets of therapeutic intervention and prevention of keloids and other fibrotic diseases.

Several treatment modalities, including surgical excision, and post-surgical adjunctive therapies such as pressure dressing, intra-lesional steroid injection, are used in the treatment of keloid. The use of GTE/EGCG is less invasive, and was proven to reduce collagen build up in keloid derived fibroblasts in vitro.

The inventors have discovered that GTE/EGCG can reduce collagen build up in keloid scar. Based on the discoveries of the present invention, the inventors have devised pharmaceutical compositions incorporating GTE/EGCG therein, Exemplary pharmaceutical compositions may include, but not limited to ointment, cream gel, spray, or dressing base for topical application or in liquid base for intralesional injection in human clinical trials.

Using the in vitro co-culture of keloid fibroblasts and human mast cells, the inventors discovered that the green tea and its major component, catechins, affects collagen synthesis in keloids. It was discovered that both GTE and EGCG significantly suppressed mast cell-stimulated type I collagen expression in keloid fibroblasts. While not intending to be bound by any particular theory, the inventors hypothesize that that the inhibitory effects of GTE and EGCG on type I collagen expression in keloid fibroblasts appeared to be mediated via the phosphatidylinositol-3-kinase (PI-3K)/Akt/mTOR (mammalian target of rapamycin) signaling pathways. These unique findings provide further understanding of the molecular mechanisms underlying the anti-fibrogenic effects of GTE and EGCG, and help to delineate further targets of therapeutic intervention and prevention of keloids and other fibrotic diseases. Based on the discoveries of the present invention, the inventors have devised pharmaceutical compositions and methods for treating keloid scars.

The following example is intended to illustrate, but not to limit, the scope of the invention. While such example is typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### Green Tea Extract and (−)-Epigallocatechin-3-gallate Inhibit Mast Cell-Stimulated Type I Collagen Expression in Keloid Fibroblasts via Blocking PI-3K/AKT Signaling Pathway

Materials and Methods

Reagents

Green Tea Extract was purchased from Pharmanex. Other products with Green Tea include ZenMed™, Replenix Green Tea, Scar Zone with Green Tea (CCA Industries, Inc.), Acne Scar System and Solutions for Acne scars.

GTE was obtained from Pharmanex Inc (Provo, Utah) and dissolved in distilled water to make a stock solution of 10 mg/mL. The purity and components of GTE were previously described (Lu et al., 2004). EGCG was purchased from Sigma (St. Louis, Mo.) and dissolved in distilled water at a concentration of 100 mmol/L and stored at −80° C. as a stock solution. PD98059, LY294002, U0126, wortmannin, SB203580, and rapamycin were purchased from Calbiochem (La Jolla, Calif.). All the inhibitors were dissolved in dimethyl sulfoxide (DMSO) with the final concentration not exceeding 0.1%. Cell viability using trypan blue exclusion was determined for both keloid fibroblasts and HMC-1 cells at the highest concentrations of inhibitors used. Mouse monoclonal antibodies against human type I collagen and β-actin were from Sigma. Mouse monoclonal antibodies against human total or phosphorylated ERK1/2 (Thr²⁰²/Tyr²⁰⁴) or Akt (Ser⁴⁷³), p38 MAPK were from New England Biolabs Inc (Beverly, Mass.). Antibodies for phosphorylated Mr. 70,000 ribosomal protein S6 kinase 1 (p70S6K) (Thr⁴²¹/Ser⁴²⁴) and eukaryotic initiation factor 4E (eIF)-binding protein 1 (4E-BP1) (Ser⁶⁵/Thr⁷⁰) were from Santa Cruz Biotechnology (Santa Cruz, Calif.). Rabbit polyclonal antibodies against human c-kit and type I collagen were from Oncogene™ Research Products (San Diego, Calif.) and Rockland Immunochemicals (Gilbertsville, Pa.), respectively. Horseradish peroxidase (HRP) conjugated secondary antibodies were from PIERCE (Rockford, Ill.). Alexa Fluor®568 conjugated rabbit anti-goat IgG and Alexa Fluor®488 conjugated goat antimouse IgG were from Molecular Probes Inc (Eugene, Oreg.). All other reagents used were analytical grade.

Cell Origin and Cell Culture

All studies have been approved by the Institutional Review Board. Primary cultures of human dermal fibroblasts were isolated by enzymatic digestion of keloid tissues obtained from patients at King Drew Medical Center (Zhang et al., 2003). All keloidal tissues collected came from untreated, primary lesions. Fibroblasts were maintained in Dulbecco's modified Eagle's medium (DMEM, Gibco, Rockville, Md.) supplemented with 10% fetal bovine serum (FBS). Cells from passage 2 through 8 were used for experiments and routinely monitored for cell proliferation, morphology and phenotype. HMC-1, a human mast cell leukemia cell line (a generous gift of J. H. Butterfield, Mayo Clinic, Rochester, Minn.), was cultured in Iscove's medium (IMDM) (Gibco) supplemented with 10% FBS, antibiotics, 2 mmol/L L-glutamine, 1.2 mmol/L α-thioglycerol (Sigma, St. Louis, Mo.). All cultures were maintained at 37° C., 5% CO₂ and 20% O₂.

Co-Culture of HMC-1 and Keloid Fibroblasts

Keloid fibroblasts (1×105/well) were seeded on the bottom of a 6-well plate and maintained at normal culturing conditions for at least 24 h. Different densities of HMC-1 cells were seeded on top of the monolayer of fibroblasts and co-cultured for different time intervals based on experimental purposes. To observe the effects of GTE and EGCG or various specific kinase inhibitors on type I collagen expression in keloid fibroblasts co-cultured with HMC-1 cells, fibroblasts at about 80% confluence were pretreated with different concentrations of GTE, EGCG, or various kinase inhibitors for 1 h followed by co-culturing with the same cell density of HMC-1 cells (1:1) under normal culturing conditions for indicated time periods. Cell lysates were prepared for Western blot analysis.

Western Blot Analyses

To determine type I collagen, and phosphorylated ERK1/2 and Akt levels, cells were solubilized in lysis buffer (50 mM Tris-HCl, pH 7.5, 150 mM NaCl, 5 mM EDTA, 200 μM Na3VO4, 50 mM NaF, 0.5% Triton X-100) supplemented with 10 mM dithiothreitol (DTT), 200 FM phenylmethylsulfonyl fluoride (PMSF) and protease inhibitor cocktails (Sigma). Total protein concentrations of whole cell lysates were determined using BioRad BCA method (PIERCE, Rockford, Ill.). Equal amounts of protein sampled from whole cell lysates were subjected to electrophoresis on 7.5%-10% sodium dodecyl sulfate (SDS)-polyacrylamide gels and electroblotted onto nitrocellulose membranes (Hybond ECL, Amersham Pharmacia). After blocking with Tris-buffered saline (TBS)/5% skim milk, the membranes were incubated overnight at 4 oC with primary antibodies against human type I collagen, total or phosphorylated ERK1/2 (Thr202/Tyr204) or Akt (Ser473). Membranes were subsequently washed, incubated with a horseradish peroxidase (HRP) conjugated secondary:antibodies (1:2000) (Pierce, Rockford, Ill.) for 1 h at room temperature, and visualized using an enhanced chemiluminescent (ECL) detection.

Measurement of COL1A1 and COL1A2 mRNA Levels by RT-PCR

Total RNA was isolated from cancer cells using TRIZOL® Reagent (Invitrogen). RT-PCR analysis of COL1A1, COL1A2 and β-actin mRNA levels was performed using the One-step RT-PCR Kit (QIAGEN, Valencia, Calif.) with primers specific to COL1A1: forward primer 5′-ATCCCACCAATCACCTGCGTA-3′ and reverse primer 5′-AACACGCTACTGCACTAGACA-3′; primers for COL1A2: forward primer 5′-CAGCAGGAGGTTTCGGCTAA-3′ and reverse primer 5′-ACCTATGCGCCTGAAACAAC-3′ (Stefanovic et al., 2005); primers for □1-actin: forward primer 5′-TCATGAAGTGTGACGTTGACATCCGT-3′ and reverse primer 5′-CCTAGAAGCATTTGCGGTGCACGATG-3′. All the primers were synthesized by Microchemical Core Facility, Norris Cancer Center of University of Southern California.

Immunofluorescence Studies

Keloid fibroblasts were seeded on 4-well Lab-TekII Chamber Slide™ System (Nalge Nunc Int., Naperville, Ill.) (1×104) and cultured for 24 h under normal condition, followed by co-culture with the same number of HMC-1 cells under the condition of direct cell-cell contact. After incubation under normoxia for 24 h, the medium was removed, and cells were fixed with cold methanol:acetone (1:1) for 15 min. The fixed cells were washed, and incubated at 4 oC overnight with a mouse monoclonal anti-human type I collagen antibody (1:100) and a rabbit polyclonal anti-human c-kit antibody (1:200). Cells were washed, incubated at room temperature for 1 h with Alexa Fluor®568 conjugated goat anti-rabbit IgG (1:2000; 0.5 μg/ml), and Alexa Fluor®®488 conjugated goat anti-mouse IgG (1:2000; 0.5 μg/ml). Slides were then viewed and photographed under a fluorescence microscope. Type I collagen expression appeared green and c-kit-positive HMC-1 cells were stained red. Cells incubated with fluoroscein-conjugated secondary antibodies in the absence of primary antibodies were used as negative control.

Statistical Analysis

Data are presented as the mean ±SD of duplicate experiments carried out for at least 3 times. One representative data set from these three independent experiments is presented where appropriate. Error bars represent SD. A paired Student's test was employed for statistical analysis, with significant differences determined as p<0.05.

Results

Mast Cells Stimulate Type I Collagen Expression in Keloid Fibroblasts In Vitro

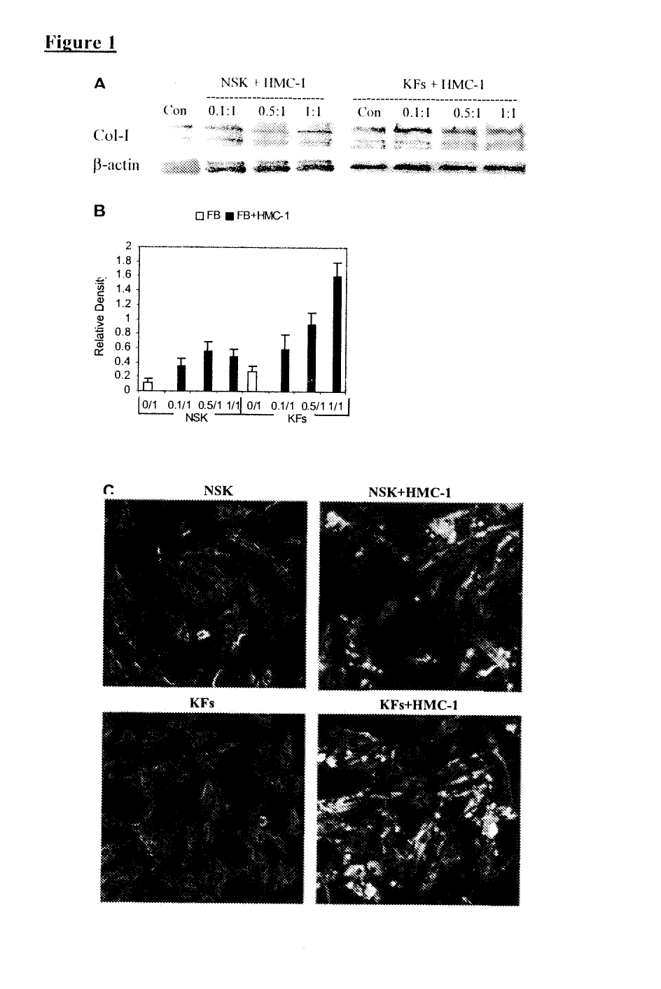

We investigated the effects of mast cells on type I collagen expression in keloid fibroblasts using an established co-culture of keloid fibroblasts and HMC-1 cells. A fixed density of normal skin or keloid fibroblasts was co-cultured with different numbers of HMC-1 cells under the condition where direct cell-cell contact is allowed. Our results from Western blot analyses showed that co-culture with HMC-1 cells led to a substantial increase in type I collagen synthesis in both normal and keloid fibroblasts, and such increase in type I collagen expression was dependent on the number of HMC-1 cells (FIGS. 1a and b). Similar results were obtained by immunofluorescence studies (FIG. 1c).

Mast Cells Stimulate Type I Collagen Expression in Keloid Fibroblasts Through Activation PI-3K/Akt and p38 MAPK Signaling Pathways

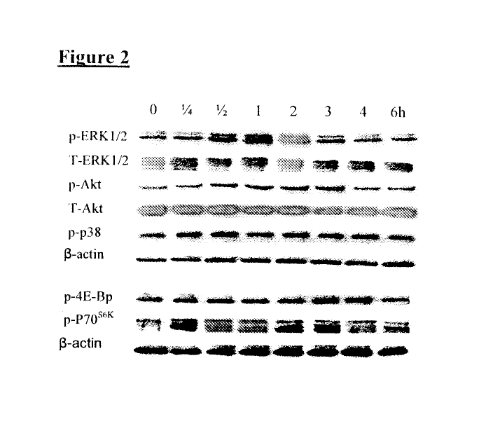

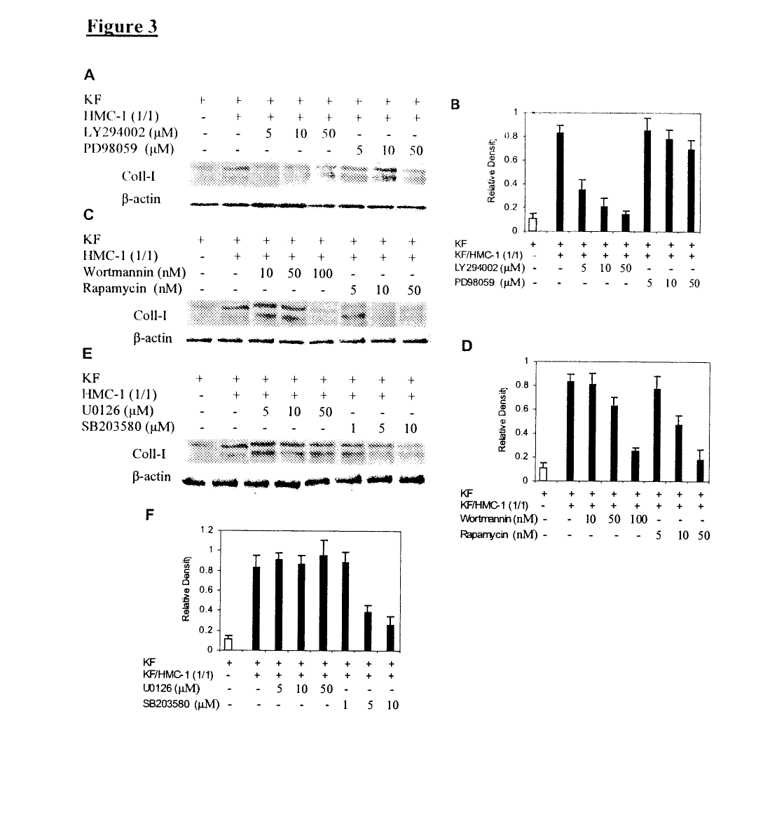

Most recently, we have demonstrated that co-culture of keloid fibroblasts and HMC-1 cells under condition of direct cell-cell contact led to the activation of both ERK1/2 and PI-3K/Akt signaling pathways (Zhang et al., 2006). Consistently, herein we also demonstrated a time-dependent increase in both phosphorylated ERK1/2 and Akt levels in keloid fibroblasts co-cultured with HMC-1 cells under normal culturing conditions, with maximal activity at 1-2 hours following co-culture (FIG. 2). In addition, our results indicated that co-culture of keloid fibroblasts with HMC-1 cells led to a time-dependent increase in phosphorylated p38 MAPK level, phosphorylated eukaryotic initiation factors (eIFs) binding protein (p-4E-BP)-1, and phosphorylated p70S6K1 levels, the two important regulatory components of the protein translational machinery (FIG. 2). These results suggested that several important signaling pathways were activated in keloid fibroblasts in response to mast cell stimulation.

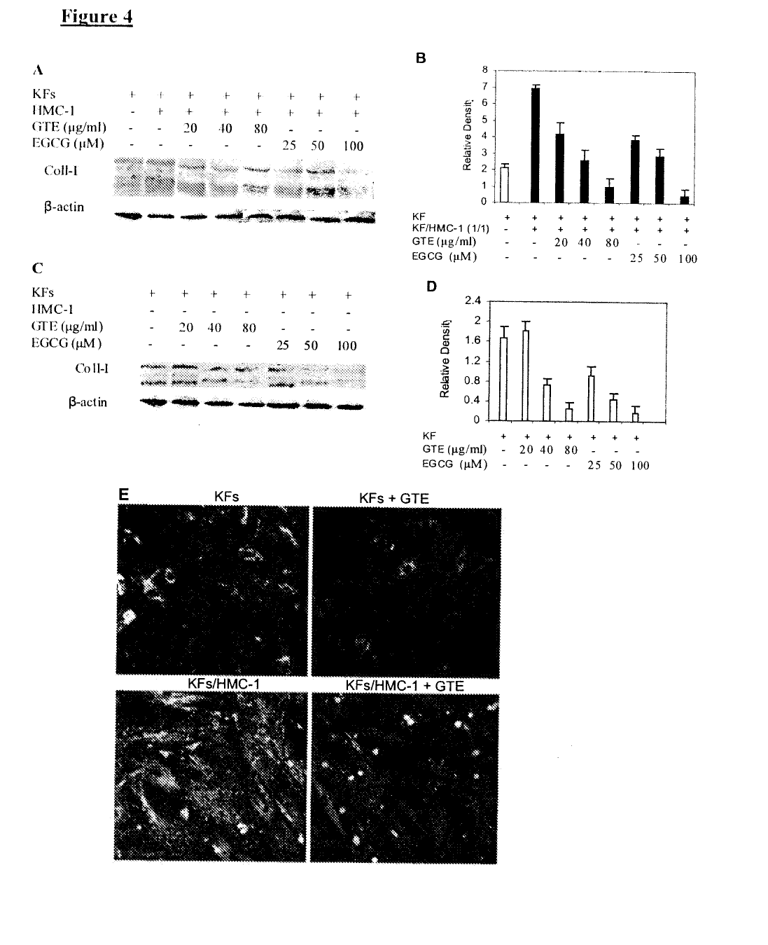

Next, we asked whether these activated signaling pathways are involved in the mast cell induced up-regulation of type I collagen expression in keloid fibroblasts. Keloid fibroblasts at about 80% confluence were pretreated with different concentrations of various protein kinase inhibitors for 1 hour followed by co-culture with the same cell density of HMC-1 cells for 24 hours under normal culturing conditions. Following treatment with the maximum dosage of various inhibitors, cell viability of both fibroblasts and HMC-1 cells was more than 95% as determined by trypan blue exclusion. Our results showed that pretreatment of keloid fibroblasts with LY294002, a specific inhibitor of PI-3K, dramatically decreased HMC-1-stimulated type collagen expression in a dose-dependent manner (p<0.05) (FIGS. 3a and b). Similar results were obtained with wortmannin, an alternative and structurally different inhibitor of PI-3K (p<0.05) (FIGS. 3c and d). Meanwhile, pretreatment of keloid fibroblasts with different concentrations of rapamycin, a specific inhibitor of mammalian target of rapamycin (mTOR), or SB203580, a specific inhibitor of p38 MAPK, also led to a dose dependent inhibition of type-I collagen expression stimulated by HMC-1 cells (p<0.05) (FIGS. 3c and d; FIGS. 3e and f). However, blocking ERK1/2 signaling pathway by pretreatment of keloid fibroblasts with PD98059 or U0126 had no obvious inhibitory effects on HMC-1-stimulated type I collagen expression (p>0.05) (FIGS. 3a and b; FIGS. 3e and f). Taken together, these observations indicated that both PI-3K/Akt and p38 MAPK signaling pathways are involved in mast cell-stimulated up-regulation of type I collagen expression in keloid fibroblasts.

GTE and EGCG Suppress HMC-1-Stimulated Type I Collagen Expression in Keloid Fibroblasts by Interfering with PI-3K/Ak Signaling Pathway

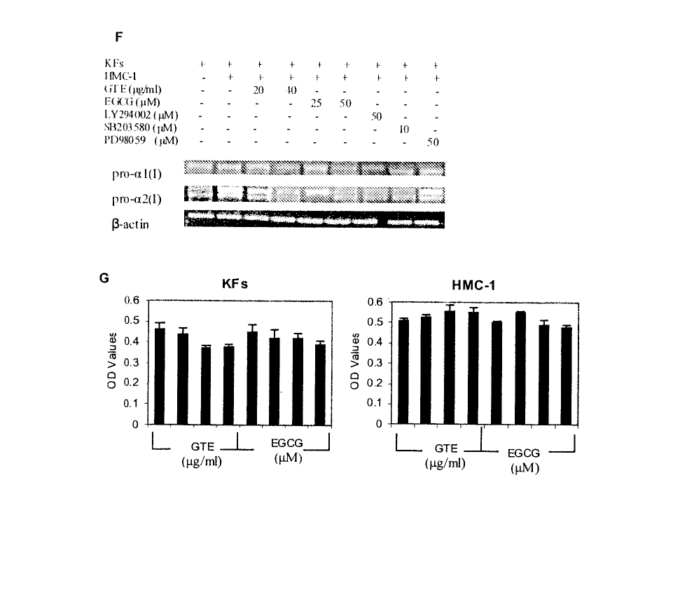

Previous studies have shown that green tea and its major catechins not only have inhibitory effects on mast cell activation (Kakegawa et al., 1985; Yamamoto et al., 1998; Li et al., 2005), but also possess anti-fibrogenic activity in some animal models (Zhong et al., 2003; Kapoor et al., 2004; Nakamuta et al., 2005). These findings prompted us to explore whether green tea extract (GTE) and one of its major catechins, (−)-epigallocatechin-3-gallate (EGCG), had any effects on mast cell-stimulated type I collagen expression in keloid fibroblasts. To this purpose, keloid fibroblasts were pretreated with different concentrations of GTE or EGCG for 1 hour followed by co-culture with the same cell density of HMC-1 cells with direct cell-cell contact for 24 hours under normal culturing conditions, and type I collagen protein and pro-COL1A1 and COL1A2 gene mRNA levels were determined by Western blot and RT-PCR, respectively. Our results showed that pretreatment with GTE or EGCG led to a dose-dependent reduction in HMC-1-stimulated type I collagen protein production and mRNA messages in keloid fibroblasts (p<0.05) (FIGS. 4a and b; FIG. 4f). Immunofluorescence studies also showed that treatment with GTE significantly attenuated type I collagen signals in keloid fibroblasts stimulated by HMC-1 cells (FIG. 4e, the lower right panel v.s. the lower left panel). To find out whether GTE and EGCG have any effects on the basal level of type I collagen in keloid fibroblasts, cells were cultured in the presence of different concentrations of GTE or EGCG for 24 hours and type I collagen levels were determined by Western blot. Our results showed that treatment with GTE and EGCG inhibited the constitutive expression of type I collagen in a dose-dependent manner (FIGS. 4c and d). Similar results were observed by immunofluorescence studies (FIG. 4e, the upper right panel v.s. the upper left panel). To rule out the possibility that the inhibitory effect of GTE and EGCG on type I collagen expression was due to cellular toxicity, cell viability was determined using MTT assay. No obvious changes in cell viability were observed in both keloid fibroblasts and HMC-1 cells after treatment with different concentrations of GTE and EGCG under normal conditions for 24 hours (FIG. 4g).

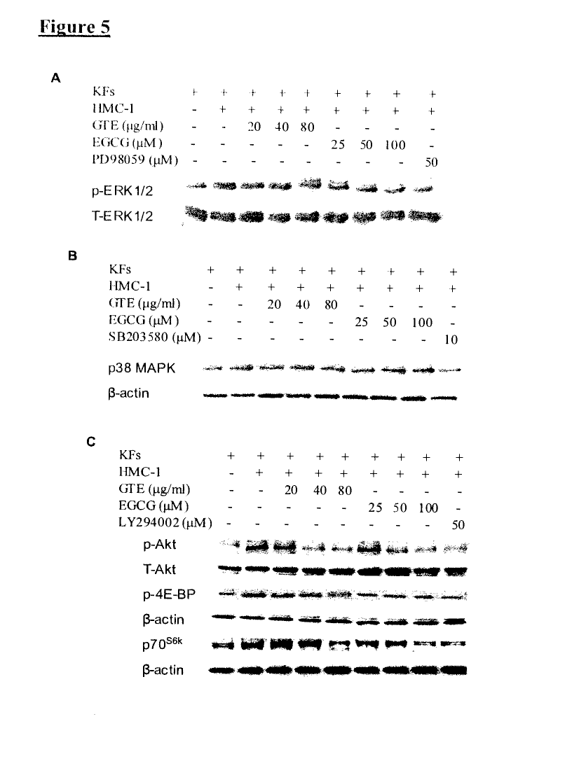

Then we explored the signaling mechanisms underlying the inhibitory effects of GTE and EGCG on HMC-1-stimulated type I collagen expression in keloid fibroblasts. As shown in FIG. 5, pretreatment of keloid fibroblasts with either GTE or EGCG did not have any obvious inhibitory effects on phosphorylated ERK1/2 and p38 MAPK levels stimulated by co-culture with HMC-1 cells (FIGS. 5a and b). On the other hand, treatment with GTE or EGCG resulted in a dose-dependent reduction in the phosphorylated Akt, p-4E-BP and p-p70S6K levels stimulated by HMC-1 cells in keloid fibroblasts (FIG. 5c), which paralleled their inhibitory effects on HMC-1-stimulated up-regulation of type I collagen expression (FIG. 4). Collectively, these data suggest that GTE and EGCG inhibited mast cell-stimulated type I collagen expression in keloid fibroblasts possibly by interfering the PI-3K/Akt signaling pathways.

Discussion

In the skin, mast cells are frequently residing in the dermis and closely associated with vessels and appendages (Metcalfe et al., 1997). Due to their ability to respond to a composite range of stimuli and release, by means of degranulation, a wide array of biologically active mediators, mast cells have been identified to play a pivotal role in many patho-physiological conditions such as innate and acquired immunity (Mekori et al., 2000), wound healing (Artuc et al., 1999; Noli et al., 2001), inflammation (Puxeddu et al., 2003; Wooley, 2003; Nigrovic et al., 2005), fibrosis (Farrel et al., 1995; Cairns et al., 1997; Gruber et al., 1997; Abe et al., 2000; Garbuzenko et al., 2002; Li et al., 2002), tumors and autoimmune diseases (Benoist et al., 2002; Ribatti et al., 2004). Previous studies have shown that mast cells undergo significant qualitative as well as quantitative changes in some chronic inflammatory diseases associated with fibrosis such as inflammatory arthritis (Wooley, 2003; Nigrovic et al., 2005), liver fibrosis (Farrel et al., 1995), chronic wounds (Huttunen et al., 2000), scleroderma (Wang et al., 2005), hypertrophic scars and keloids (Kischer et al., 1978; Craig et al., 1986; Smith et al., 1987; Lee et al., 1995).

Previously, several studies have shown that co-culture with mast cells promote type I collagen synthesis in fibroblasts (Cairns et al., 1997; Gruber et al., 1997; Abe et al., 2000; Garbuzenko et al., 2002). Consistently, our current studies demonstrate that co-culture with mast cells substantially stimulates type I collagen synthesis in keloid fibroblasts (FIG. 1). However, the associated signaling mechanisms remain largely unknown. Most recently, our group has reported that direct co-culture with HMC-1 cells leads to transient activation of ERK1/2 and PI-3K/Akt signaling pathways in keloid fibroblasts (Zhang et al., 2006). The present experiment has extended our previous observation and found that co-culture with HMC-1 cells activates not only ERK1/2 and PI-3K/Akt signaling pathways but also p38 MAPK pathways (FIG. 2). This finding agrees well with previous report that co-culture of mast cell and lung fibroblast with direct cell-cell interaction induces IL-6 production in a concentration- and time-dependent manner by activating p38 MAPKs (Fitzgerald et al., 2004). In addition, our results also indicate that co-culture with HMC-1 cells increases the phosphorylated levels of p4E-BP and pp 70S6K (FIG. 2), two important regulatory components of protein translational machinery that are downstream targets of PI-3K/Akt/mTOR signaling pathways. Previously, several studies have demonstrated the involvement of ERK1/2, PI-3K/Akt/mTOR, and p38 MAPK signaling pathways in the up-regulation of type I collagen expression in dermal fibroblasts (Lim et al., 2003; Asano et al., 2004; Shegogue et al., 2004; Ihn et al., 2005). In this experiment, we have demonstrated for the first time that treatment with specific inhibitors of PI-3K, mTOR and p38 MAPK significantly inhibits mast cell-stimulated type I collagen production in keloid fibroblasts while treatment with specific inhibitors of ERK1/2 has no obvious inhibitory effects (FIG. 3), thus suggesting that mast cells stimulate type I collagen expression in keloid fibroblasts via PI-3K/Akt/mTOR and p38 MAPK signaling pathways.

Green tea extract (GTE) and its major polyphenolic components known as catechins have long been proved to possesses various pharmacological activities, and consumption of tea has been associated with many health benefits including the prevention of cancer and heart diseases (Yang et al., 2002). Recent studies have shown that some components of green tea can inhibit mast cell activation (Kakegawa et al., 1985; Yamamoto et al., 1998; Li et al., 2005) and affect ECM metabolism and remodeling in several experimental models (Zhong et al., 2003; Kapoor et al., 2004; Nakamuta et al., 2005). In this study, we reported for the first time that GTE and EGCG, one of the major catechins of green tea, have strong inhibitory effects on mast cell-stimulated type I collagen production in keloid fibroblasts (FIG. 4). Moreover, our results indicate that treatment with GTE or EGCG remarkably decreases mast cell-stimulated up-regulation of phosphorylated Akt, p4E-BP and p-p70S6K₁ levels, but has no obvious inhibitory effects on the up-regulated levels of phosphorylated ERK1/2 and p38 MAPK in keloid fibroblasts (FIG. 5). These results suggest that GTE or EGCG inhibit mast cell-stimulated type collagen production mainly by interfering the PI-3K/Akt signaling pathways. Therefore, our findings have provided further evidence that GTE and EGCG harbor potential anti-fibrogenic activity.

In summary, the present study has demonstrated that mast cells can stimulate type I collagen production via activating multiple signaling pathways in keloid fibroblasts. Keloids, a chronic fibroproliferative disease, are characterized by excessive collagen deposition, and, notoriously, to be prone to recurrence. Even though a number of treatment modalities have been employed to overcome keloid or to relieve its symptoms, there is no one modality that is always successful (Poochareon et al., 2003; Kelly, 2004; Burd et al., 2005). Herein, we have also demonstrated for the first time that both GTE and EGCG significantly inhibit mast cell-stimulated type I collagen expression by interfering with PI-3k/Akt signaling pathway in keloid fibroblasts. Therefore, our unique findings have provided further evidence for the molecular mechanisms of keloid pathogenesis and identified a therapeutic potential of green tea for the intervention and prevention of keloids and other fibrotic diseases

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

Abe M, Kurosawa M, Ishikawa O, Miyachi Y (2000) Effect of mast cell-derived mediators and mast-cell-related neutral proteases on human dermal fibroblast proliferation and type I collagen production. J Allergy Clin Immunol 106: S78-84.
Amadeu T, Braune A, Mandarim-de-Lacerda C, Porto L C, Desmouliere A, Costa A (2003) Vascularization pattern in hypertrophic scars and keloids: a stereological analysis. Pathol Res Pract 199: 469-73.
Artuc M, Hermes B, Stechelings U M, Grutzkau A, Henz B M (1999) Mast cells and their mediators in cutaneous wound healing-active participants or innocent bystanders? Exp Dermatol 8: 1-16.
Asano Y, Ihn H, Yamane K, Jinnin M, Mimura Y, Tamaki K (2004) Phosphatidylinositol 3-kinase is involved in □2 (I) collagen gene expression I noral and scleroderma fibroblasts. J mmunol 172: 7123-35.
Baliga M S, Meleth S, Katiyar S K (2005) Growth inhibitory and antimetastatic effect of green tea polyphenols on metastasis-specific mouse mammary carcinoma 4T1 cells in vitro and in vivo systems. Clin Cancer Res 11: 1918-27.
Benoist C, Mathis D (2002) Mast cells in autoimmune disease. Nature 420: 875-8.
Burd A and Huang L (2005) Hypertrophic response and keloid diathesis: two very different forms of scar. Plast Reconstr Surg 116: 150-7e.
Cairns J A, Walls A F (1997) Mast cell tryptase stimulates the synthesis of type I collagen in human lung fibroblasts. J Clin Invest 99: 1313-21.
Caturla N, Vera-Samper Villalain J, Reyes Mateo C, Micol V (2003) The relationship between the antioxidant and the antibacterial properties of galloylated catechins and the structure of phospholipids model membranes. Free Rad Biol Med 34: 648-62.
Chan MM-Y, Dunne F, Ho C-T, Huang H-I (1999) Inhibition of inducible nitric oxide synthase gene expression and enzyme activity by epigallocatechin gallate, a natural product from green tea. Biochem Pharmacol 54: 1281-6.
Choi K L, Claman H N (1987) Mast cells, fibroblasts and fibrosis: New clues to the riddle of mast cells. Immunol Res 6: 145-152.
Craig S S, DeBlois G, Schwartz L B (1986) Mast cells in human keloid, small intestine, and lung by an immunoperoxidase technique using a murine monoclonal antibody against tryptase. Am J Pathol 124: 427-35.
Crouvezier S, Powell B, Keir D, Yaqoob P (2000) The effects of phenolic components of tea on the production of pro- and anti-inflammatory cytokines by human leukocytes in vitro. Cytokine 13: 280-6.
Ehrlich H P, Desmouliere A, Diegelmann R F, Cohen I K, Compton C C, Garner W L et al. (1994) Morphological and immunochemical differences between keloid and hypertrophic scar. Am J Pathol 145: 105-13.
Farrel D J, Hines J E, Walls A F, Kelly P J, Bennett M K, Burt A D (1995) Intrahepatic mast cells in chronic liver diseases. Hepatology 22: 1175-81.
Fassina G, Vene R, Morini M, Minghelli S, Benelli R, Noonan D M, Albini A (2004) Mechanisms of inhibition of tumor angiogenesis and vascular tumor growth by epigallocatechin-3-gallate. Clin Cancer Res 10: 4865-73.
Fitzgerald S M, Lee S A, Hall H K, Chi D S, Krishnaswamy G (2004) Human lung fibroblasts express interleukin-6 in response to signaling after mast cell contact. Am J Respir Cell Mol Biol 30: 585-93.
Garbuzenko E, Nagler A, Pickholtz D, Gillery P, Reich R, Maquart F-X, Levi-Schaffer F (2002) Human mast cells stimulate fibroblast proliferation, collagen synthesis and lattice contraction: a direct role for mast cells in skin fibrosis. Clin Exp Allerg 32: 237-46.
Ghosh A K (2002) Factors involved in the regulation of type I collagen gene expression: implication in fibrosis. Exp Biol Med 227: 301-14.
Gruber B L, Kew R R, Jelaska A, Marchese M J, Garlick J, Ren S et al. (1997) Human mast cells activate fibroblasts: tryptase is a fibrogenic factor stimulating collagen messenger ribonucleic acid synthesis and fibroblast chemotaxis. J Immunol 158: 2310-7.
Han M K (2003) Epigallocatechin gallate, a constituent of green tea, suppresses cytokine induced pancreatic □-cell damage. Exp Mol Med 35: 136-9.
Huttunen M, Aalto M L, Harvima, R J, Horsmanheimo M, Harvima I T (2000) Alterations in mast cells showing tryptase and chymase activity in epithelialization and chronic wounds. Exp Dermatol 9: 258-65.
Ihn H, Yamane K, Tamaki K (2005) Increased phosphorylation and activation of mtogen-activated protein kinase p38 in scleroderma fibroblasts. J Invest Dermatol 125: 247-55.

Kakegawa H, Matsumoto H, Endo K, Satoh T, Nonaka G, Nishioka I (1985) Inhibitory effects of tannins on hyaluronidase activity and on the degranulation from rat mesentery mast cells. Chem Pharm Bull 33: 5079-82.
Kapoor M, Howard R, Hall I, Appleton I (2004) Effects of epicatechin gallate on wound healing and scar formation in a full thickness incisional wound healing model in rats. Am J Pathol 165: 299-307.
Katiyar S K, Matsui M S, Elmets C A, Mukhtar H (1999) Polyphenolic antioxidant epigallocatechin-3-gallate from green tea reduces UVB-induced inflammatory responses and infiltration of leukocytes in human skin. Photochem Photobiol 69: 148-53.
Kelly A P (2004) Medical and surgical therapies for keloids. Dermatol Ther 17: 212-8.
Kim Y S, Jhon D Y, Lee K Y (2004) Involvement of ROS and JNK 1 in selenite-induced apoptosis in Chang liver cells. Exp Mol Med 36: 157-64.
Kischer C W, Bunce H, Shetlah M R (1978) Mast cell analyses in hypertrophic scars, hypertrophic scars treated with pressure and mature scars. J Invest Dermatol 70: 355-61.
Lee J Y, Yang C C, Chao S C, Wong T W (2004) Histopathological differential diagnosis of keloid and hypertrophic scar. Am J Dermatopathol 26: 379-84.
Lee Y S, Vijayasingam S (1995) Mast cells and myofibroblasts in keloid: a light microscopic, immunohistochemical and ultrastructural study. Ann Acad Med Singapore 24: 902-5.
Li C Y, Baek J Y (2002) Mastocytosis and fibrosis: role of cytokines. Int Arch Allergy Immunol 127: 123-6.
Li G Z, Chai O H, Song C H (2005) Inhibitory effects of epigallocatechin gallate on compound 48/80-induced mast cell activation and passive cutaneous anaphylaxis. Exp Mol Med 37: 290-6.
Lim I J, Phan T T, Tan E K, Nguyen T-T, Tran E, Longaker M T, Song C, Lee S T, Huynh H T (2003) Synchronous activation of ERK and phosphatidylinositol 3-kinase pathways is required for collagen and extracellular matrix production in keloids. J Biol Chem 278: 40851-6.
Lu Q Y, Jin Y S, Pantuck A, Zhang Z F, Heber D, Belldegrun A et al. (2005) Green tea extract modulates actin remodeling via Rho activity in an in vitro multistep carcinogenic model. Clin Cancer Res 11: 1675-83.
Mekori Y A, Metcalfe D D (2000) Mast cells in innate immunity. Immunol Rev 173: 131-40.
Metcalfe D D, Baram D, Mekori Y A (1997) Mast cells. Physiol Rev 77: 1033-70.
Myllyharju J, Kivirikko K I (2001) Collagens and collagen-related diseases. Ann Med 33: 7-21.
Nakamuta M, Higashi N, Kohjima M, Fukushima M, Ohta S, Kotoh K, Kobavashi N, Enjoji M (2005) Epigallocatechin-3-gallate, a polyphenol component of green tea, suppresses both collagen production and collagenase activity in hepatic stellate cells. Int J Mol Med 16: 677-81.
Niessen F B, Spauwen P H M, Schalkwijk J, Moshe K K (1999) On the nature of hypertrophic scars and keloids: a review. Plast Reconstr Surg 104: 1435-58.
Nigrovic P A, Lee D M (2005) Mast cells in inflammatory arthritis. Arthritis Res Ther 7: 1-11.
Noli C, Miolo A (2001) The mast cell in wound healing. Vet Dermatol 12: 303-13.
Poochareon V N, Berman B (2003) New therapies for the management of keloids. J Craniofacial Surg 14: 654-7.
Puxeddu I, Piliponsky A M, Bachelet I, Levi-Schaffer F (2003) Mast Cells in Allergy and beyond. Int J Biochem & Cell Biol 35: 1601-7.
Raghow R (1994) The role of extracellular matrix in postinflammatory wound healing and fibrosis. FASEB J 8: 823-31.
Ribatti D, Crivellatow E, Roccaroz A M, Riaz R, Vaccaz A (2004) Mast cell contribution to angiogenesis related to tumour progression. Clin Exp Allergy 34: 1660-4.
Rockwell W B, Cohen I K, Ehrlich H P (1989) Keloids and hypertrophic scars: a comprehensive review. Plast Reconstr Surg 84: 827-37.
Shegogue D, Trojanowska M (2004) Mammalian target of rapamycin positively regulates collagen type I production via a phosphatidylinositol 3-kinase independent pathway. J Biol Chem 279: 23166-75.
Smith C J, Finn M C (1987) The possible role of mast cells (allergy) in the production of keloid and hypertrophic scarring. J Burn Care Rehabilitation 8: 126-31.
Stefanovic L, Stephens C E, Boykin D, Stefanovic B (2005) Inhibitory effect of dicationic diphenylfurans on production of type I collagen by human fibroblasts and activated hepatic stellate cells. Life Sci 76: 2011-26.
Tuan T L, Nichter L S (1998) The molecular basis of keloid and hypertrophic scar formation. Mol Med Today 4: 19-24.
Wang H-W, Tedla N, Hunt J E, Wakefield D, McNeil H P (2005) Mast cell accumulation and cytokine expression in the tight skin mouse model of scleroderma. Exp Dermatol 14: 295-302.
Wooley D E (2003) The mast cell in inflammatory arthritis. New Eng J Med 348: 1709-11.
Yamamoto M M, Kawahara H, Matsuda N, Nesumi K, Sano M, Tsuji K, Kawakami Y, Kawakami T (1998) Effects of tea infusions of various varieties or different manufacturing types on inhibition of mouse mast cell activation. Biosci Biotechnol Biochem 62: 2277-9.
Yang C S, Maliakal P, Meng X (2002) Inhibition of carcinogenesis by tea. Annu Rev Pharmacol Toxicol 42: 25-54.
Zhang Q Z, Oh C K, Messadi D V, Duong H S, Kelly A P, Soo C, Wang L, Le A (2006) Hypoxia-induced HIF-1□ accumulation is augmented in a co-culture of keloid fibroblasts and human mast cells: involvement of ERK1/2 and PI-3K/Akt. Exp Cell Res 312: 145-55.
Zhang Q Z, Wu Y D, Ann D K, Messadi D V, Tuan T L, Kelly A P, Bertolami C N, Le A (2003) Mechanisms of hypoxic regulation of plasminogen activator inhibitor-1 gene expression in keloid fibroblasts. J Invest Dermatol 121: 1005-12.
Zhong Z, Froh M, Lehnert M, Schoonhoven R, Yang L, Lind H, Lemasters J J, Thurman R G (2003) Polyphenols from Camellia sinenesis attenuate experimental cholestasis-induced liver fibrosis in rats. Am J Physiol Gastrointest Liver Physiol 285: G1004-13.

What is claimed is:

1. A pharmaceutical composition for treating keloid scars, comprising:
substantially pure epigallocatechin-3-gallate.

2. The composition of claim 1, further comprises green tea extract.

3. A method for preventing keloid scars, comprising:
applying a pharmaceutical composition containing pharmaceutically effective amount of green tea extract to a fresh skin wound of a subject,
whereby the composition is capable of inhibiting expression of collagen in keloid fibroblasts.

4. The method of claim 3, wherein the pharmaceutical composition comprises substantially pure epigallocatechin-3-gallate.

5. The method of claim 3, wherein applying the pharmaceutical composition comprises applying the composition to the wound at regular intervals until the wound is healed.

6. A method for treating keloid scars, comprising:
removing existing keloid scars;
applying a pharmaceutical composition according to claim 1; and
allowing the wound to heal.

7. The method of claim 6, wherein the removing step is performed by laser surgery.

8. The method of claim 6, wherein the removing step is performed by conventional surgery with a surgical knife.


SEQUENCE LISTING (6 sequences)

SEQ ID NO: 1 - DNA, 21 bp, Artificial
  Synthetic oligonucleotide
atcccacgaatcacctgcgta

SEQ ID NO: 2 - DNA, 21 bp, Artificial
  Synthetic oligonucleotide
aacacgctactgcactagaca

SEQ ID NO: 3 - DNA, 20 bp, Artificial
  Synthetic oligonucleotide
cagcaggaggtttcggctaa

SEQ ID NO: 4 - DNA, 20 bp, Artificial
  Synthetic oligonucleotide
acctatgcgcctgaaacaac

SEQ ID NO: 5 - DNA, 26 bp, Artificial
  Synthetic oligonucleotide
tcatgaagtctgacgttgacatccgt

SEQ ID NO: 6 - DNA, 26 bp, Artificial
  Synthetic oligonucleotide
cctagaagcatttgcggtgcacgatg